(19) United States
(12) Patent Application Publication
(10) Pub. No.: US 2016/0128589 A1
(43) Pub. Date: May 12, 2016

(54) NERVOUS SYSTEM INTERFACE DEVICE

(71) Applicant: University of Utah Research Foundation, Salt Lake City, UT (US)

(72) Inventor: Massood Tabib-Azar, Salt Lake City, UT (US)

(21) Appl. No.: 14/938,634

(22) Filed: Nov. 11, 2015

Related U.S. Application Data
(60) Provisional application No. 62/078,109, filed on Nov. 11, 2014.

(51) Int. Cl. A61B 5/04; A61B 5/0478; A61B 5/00

(52) U.S. Cl. CPC A61B 5/04001; A61B 5/0004; A61B 5/0478; A61B 5/6868; A61B 2560/0204

(57) ABSTRACT

A nervous system interface device can include a flexible active layer, a power coil, a communication module, and an antenna. More specifically, the flexible active layer can included a plurality of electrodes that are positioned to take neural measurements of the nervous system. The power coil can be electrically coupled to the flexible active layer and can be configured to receive wireless power from a power transfer device. The communication module can be electrically coupled to the flexible active layer and can be configured to receive power from the power coil. The antenna can also be adapted to wirelessly communicate neural measurement information to another device (e.g. an external computing device, receiver or the like). The nervous system interface device has two functional configurations which include a rolled and an unrolled configuration. In the rolled configuration the nervous system electrode device is rolled along a longitudinal axis for insertion into a hypodermic needle. In the unrolled configuration the nervous system electrode device is substantially flat. These nervous system electrode devices can dramatically improve access to brain function information, increase brain-machine interface functionality, and decrease foreign body response.

### RELATED APPLICATION

This application claims priority to U.S. Provisional Application No. 62/078,109 filed on Nov. 11, 2014, which is herein incorporated by reference.

### GOVERNMENT INTEREST

This invention was made with government support under Grant No. CBET 0933234 awarded by U.S. National Science Foundation. The government has certain rights to the invention.

### BACKGROUND

A number of approaches exist for monitoring and measuring electrical signals or magnetic signals of animal brains and/or human brains using different neural sensor probes. A non-invasive approach to measuring electrical signals or magnetic signals of animal or human brains can be to use electroencephalography (EEG), magnetoencephalography (MEG), or functional magnetic resonance imaging (fMRI). In another example, an invasive approach can be used by placing sensors or probes, such as electrodes, in a space between the skin and the skull of a patient or below the skull, such as directly atop the membrane of the brain or penetrating into the brain.

With respect to invasive approaches, the probes can be located below the skull and can rest on top of the surface of the brain, e.g. electrocorticographic (ECoG) arrays, or the probes can be located below the skull while individual sensor elements penetrate brain tissue, such as in multi-electrode arrays (MEAs). One advantage of the invasive approaches, such as using ECoG arrays and MEAs, is that neural measurements taken invasively provide more detailed information about neural circuit functions than the measurements taken using non-invasive approaches. For example, MEAs enable extraction of neural system signals with an increased spatial resolution and temporal resolution due to each microelectrode of the array reporting measurement information of single neural cells. The measurement information can be retrieved from multiple neurons using the MEAs to monitor neural system signals for the motion of an arm, hand, or finger.

While invasive measures provide more detailed information, such measures also require that the subject's protective structures be pierced and opened. These protective structures include the outer skin of a human or animal which protects against environmental contaminants and, when taking some central nervous system measurements, the skull which forms the protective cavity for the brain. Cranial sensors, such as ECoG multi-electrodes and MEA multi-electrodes, are implanted below the skin and can be implanted below the skull. The ECoG multi-electrodes and MEA multi-electrodes have wires exiting the skin and when the electrodes are implanted under the skull these wires also exit the skull beneath the skin. These exit points require that the protective structures remain open and have safety and longevity limits due to an increased risk of infection and a decreased mobility of the patient.

### SUMMARY

In one embodiment, a nervous system interface device is presented. The nervous system interface device can comprise a flexible active layer, a power coil, a communication module, and an antenna. More specifically, the flexible active layer can include a plurality of electrodes that are positioned to take neural measurements of a nervous system. The power coil can be electrically coupled to the flexible active layer and can be configured to receive wireless power from a power transfer device. The communication module can be electrically coupled to the flexible active layer and can be configured to receive power from the power coil. The antenna can be adapted to wirelessly communicate neural measurement information to another device (e.g. an external computing device, a receiver, or the like). The nervous system interface device has two functional configurations which include a rolled and an unrolled configuration. The rolled configuration allows the nervous system interface device to be rolled along a longitudinal axis for insertion into a hypodermic needle. The unrolled configuration permits the electrode device to lie substantially flat.

In another embodiment, an intracranial electrode system is presented. The intracranial electrode system can comprise a shaft, an opening at the end of the shaft, and an intracranial electrode. The shaft can be sized and shaped to be inserted into a cranial opening of a patient. The opening at the end of the shaft can be sized and shaped to be positioned subcutaneously adjacent to a patient's cranial membrane. The opening at the end of the shaft can also be sized and shaped to receive the intracranial electrode. The intracranial electrode can be sized and shaped to be rolled along a longitudinal axis of the intracranial electrode and can be adapted to be inserted into the opening of the shaft.

In a further embodiment, a method of taking a neural measurement is presented. The method can comprise several steps. First, a flexible electrode that is sized and shaped to be rolled along a longitudinal axis can be inserted in a delivery shaft. Then the delivery shaft can be inserted into an opening in a patient. The flexible electrode can then be discharged from the delivery shaft into the opening of the patient. The method further comprises optionally transferring wireless power from a power transfer device to the flexible electrode. The flexible electrode can then be used to take a neural measurement. The neural measurement can be wirelessly communicated to a receiving device.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1a depicts a front view of an injectable or implantable nervous system interface device in accordance with an example;

FIG. 1b depicts a back view of the injectable implantable nervous interface device of FIG. 1a;

FIG. 1c depicts a side view of the injectable implantable nervous system interface device of FIG. 1a;

FIG. 1d depicts a side view of the nervous system interface device of FIG. 1c rotated 90 degrees;

FIG. 2a depicts a lateral arrangement of an injectable implantable nervous system interface device that can include a flexible coil in accordance with an example;

FIG. 2b depicts a plurality of injectable implantable nervous system interface devices receiving wireless power from a radio frequency (RF) coil of another device to power the injectable implantable nervous system interface devices in accordance with an example;

FIG. 3 shows a porous texture or sieves in a portion of a flexible coil in accordance with an example;

FIG. 4a depicts an injectable implantable nervous system interface device in an unrolled configuration in accordance with an example;

FIG. 4b depicts an injectable implantable nervous system interface device in a rolled configuration in accordance with an example;

FIG. 5 depicts an injectable implantable nervous system device in a rolled configuration partially inserted into a hypodermic needle in accordance with an example;

FIG. 6 illustrates several injectable implantable nervous system devices embedded to record signals in multiple brain areas in accordance with an example;

FIG. 7 illustrates a table of electrode devices with different configurations of electrode arrays in accordance with an example;

FIG. 8a illustrates a low power frequency-selectable and addressable FM transmitter for neural recording in accordance with an example;

FIG. 8b illustrates a graph of a frequency versus voltage level of the transmitter in accordance with an example;

FIG. 9a depicts a graph of neural signals in accordance with an example; and

FIG. 9b depicts a graph of neural signals in accordance with an example.

These drawings are provided to illustrate various aspects of the invention and are not intended to be limiting of the scope in terms of dimensions, materials, configurations, arrangements or proportions unless otherwise limited by the claims. Reference will now be made to the exemplary embodiments illustrated, and specific language will be used herein to describe the same. It will nevertheless be understood that no limitation of the scope of the invention is thereby intended.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### DEFINITIONS

In describing and claiming the present invention, the following terminology will be used.

The singular forms “a,” “an,” and “the” include plural referents unless the context clearly dictates otherwise. Thus, for example, reference to “an electrode” includes reference to one or more electrodes.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

In this disclosure, “comprises,” “comprising,” “containing” and “having” and the like can have the meaning ascribed to them in U.S. Patent law and can mean “includes,” “including,” and the like, and are generally interpreted to be open ended terms. The term “consisting of” is a closed term, and includes only the devices, methods, compositions, components, structures, steps, or the like specifically listed, and that which is in accordance with U.S. Patent law. “Consisting essentially of” or “consists essentially” or the like, when applied to devices, methods, compositions, components, structures, steps, or the like encompassed by the present disclosure, refers to elements like those disclosed herein, but which may contain additional structural groups, composition components, method steps, etc. Such additional devices, methods, compositions, components, structures, steps, or the like, etc., however, do not materially affect the basic and novel characteristic(s) of the devices, compositions, methods, etc., compared to those of the corresponding devices, compositions, methods, etc., disclosed herein. In further detail, “consisting essentially of” or “consists essentially” or the like, when applied to devices, methods, compositions, components, structures, steps, or the like encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments. When using an open ended term, like “comprising” or “including,” it is understood that direct support should be afforded also to “consisting essentially of” language as well as “consisting of” language as if stated explicitly.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Reference throughout this specification to “an example” means that a particular feature, structure, or characteristic described in connection with the example is included in at least one embodiment of the present invention. Thus, appearances of the phrases “in an example” in various places throughout this specification are not necessarily all referring to the same embodiment.

Furthermore, the described features, structures, or characteristics can be combined in any suitable manner in one or more embodiments. In the following description, numerous specific details are provided, such as examples of layouts, distances, network examples, etc., to provide a thorough understanding of embodiments of the invention. One skilled in the relevant art will recognize, however, that the invention can be practiced without one or more of the specific details, or with other methods, components, layouts, etc. In other instances, well-known structures, materials, or operations are not shown or described in detail to avoid obscuring aspects of the invention.

As used herein, the term “at least one of” is intended to be synonymous with “one or more of.” For example, “at least one of A, B and C” explicitly includes only A, only B, only C, or combinations of each.

As will be apparent to those of skill in the art upon reading this disclosure, each of the individual embodiments described and illustrated herein has discrete components and features that may be readily separated from or combined with the features of any of the other several embodiments without departing from the scope or spirit of the present disclosure. Any recited method can be carried out in the order of events recited or in any other order that is logically possible.

It is noted in the present disclosure that when describing the systems or methods, individual or separate descriptions are considered applicable to one another, whether or not explicitly discussed in the context of a particular example or embodiment. For example, in discussing a particular system per se, the method embodiments are also inherently included in such discussions, and vice versa.

Reference will now be made to the exemplary embodiments illustrated, and specific language will be used herein to describe the same. It will nevertheless be understood that no limitation of the scope of the technology is thereby intended. Additional features and advantages of the technology will be apparent from the detailed description which follows, taken in conjunction with the accompanying drawings, which together illustrate, by way of example, features of the technology.

Presented herein is a nervous system interface device. The nervous system interface device can comprise a flexible active layer, a power coil, a communication module, and an antenna. The flexible active layer can include a plurality of electrodes that can be positioned to take neural measurements of a nervous system. The power coil can be electrically coupled to the flexible active layer and can be configured to receive wireless power from a power transfer device. The communication module can be electrically coupled to the flexible active layer and can be configured to receive power from the power coil. The antenna can be adapted to wirelessly communicate neural measurement information to another device (e.g. an external computing device, a receiver, or the like). The nervous system interface device has two functional configurations which include a rolled and an unrolled configuration. The rolled configuration allows the nervous system interface device to be rolled along a longitudinal axis for insertion into a hypodermic needle. The unrolled configuration permits the electrode device to lie substantially flat. The nervous system interface device can be used to measure either central nervous system activity or peripheral nervous system neural activity. The central nervous system typically has more stringent requirements than peripheral nervous system, largely due to physical access to tissue. As a general guideline, electrodes targeted to peripheral nerves can be larger than CNS, which also results in a stronger signal.

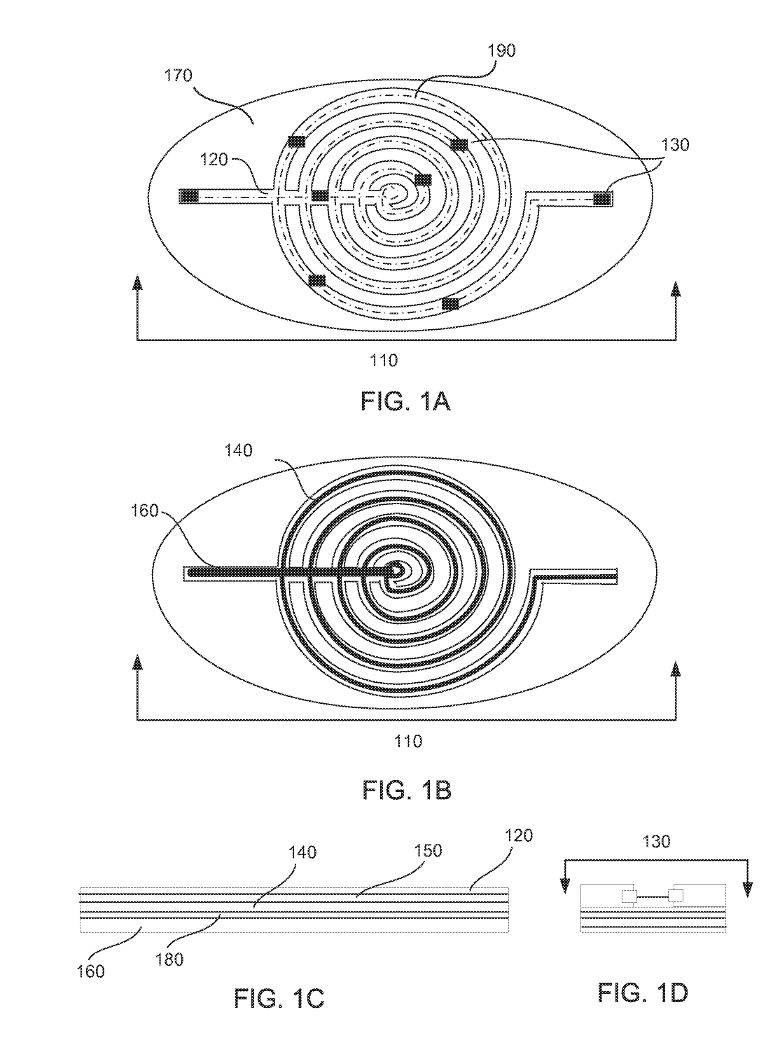

FIGS. 1a-1d depict several views of an injectable or implantable nervous system interface device 110. In one embodiment, the implantable nervous system interface device 110 can include a flexible active layer 120 with a plurality of electrodes 130. A power coil 140 and communication module 150 can be coupled to the flexible active layer. The nervous system interface device can further comprise an antenna 160 and optionally a flexible substrate 170 and a power generation and storage device 180.

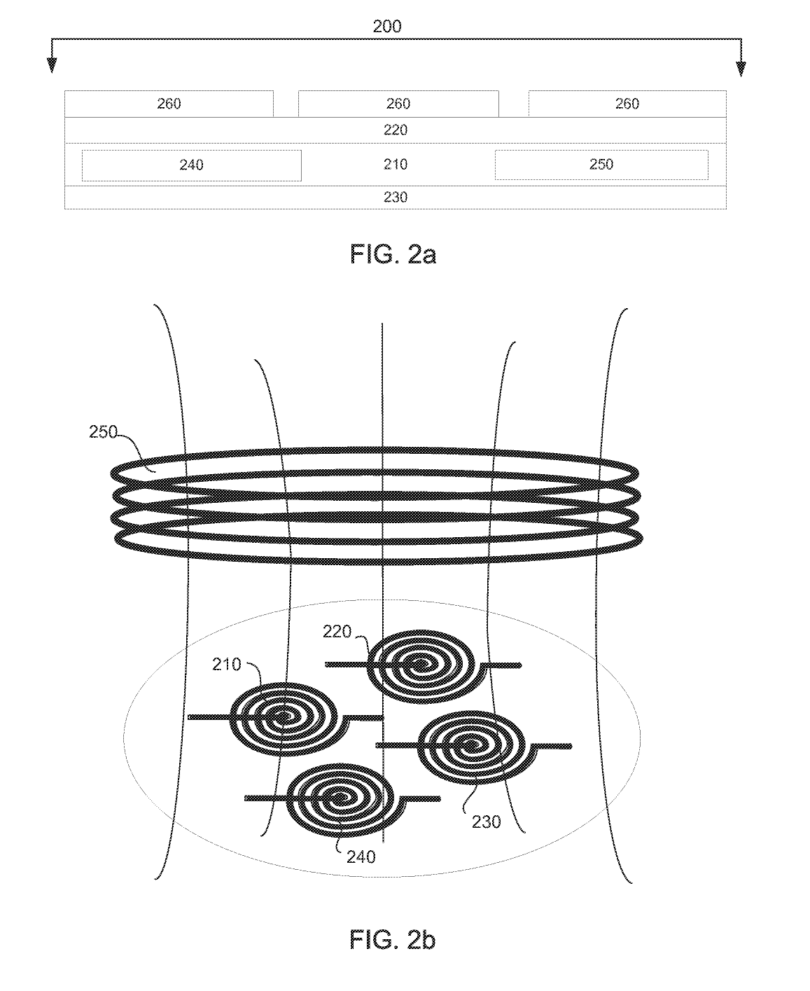

FIG. 2a depicts another embodiment of the nervous system interface device 200 that can include a flexible coil 210 as a material substrate. FIG. 2a further illustrates that the nervous system interface device 200 can include a plurality of electrodes 220. The nervous system interface device 200 can include a power coil 230 coupled to the bottom surface of the flexible coil 210 configured to receive wireless power from a power transfer device. In one embodiment, the nervous system interface device 200 can include a communication module 240 coupled to the flexible coil 210 that is configured to receive power from the power coil and includes an antenna configured to wirelessly communicate neural measurement information to another device. In another embodiment, the nervous system interface device 200 can include a flexible polymeric battery 250 that can be coupled to the flexible coil 210. In another embodiment, the flexible polymeric battery 250 can be configured to receive energy from the power coil 230 and store energy in one or more cells of the flexible polymeric battery 250. The electrodes 220 can also be coated with a polymer coating 260 where individual exposed electrodes active sites are etched through the polymer coating.

Turning now to the details of the nervous system interface device. The flexible active layer houses the plurality of electrodes. The flexible active layer can exist in a variety of shapes. In one embodiment the flexible active layer has a coiled shape. The coiled shape can be a rounded or a polygonal coil. In other embodiments, the flexible active layer can be circular, square, triangular, or polygonal. The flexible active layer can generally be a thin support structure. Although thicknesses can vary, the support structure can often have a thickness from 1 μm to 25 μm, and in some cases from 200 μm to 300 μm. Although as a general guideline, a thinner support layer allows for increased flexibility, the support layer can also mechanically support the electrodes and associated electronics, power storage and communication components (inductor, antenna, etc.).

In one embodiment, the flexible active layer can be about 100 μm in diameter. In other embodiments, the flexible active layer can be about 10 μm to about 500 μm in diameter (or largest planar dimension). The area of the flexible active layer can be from about 10 μm by 10 μm to about 100 μm by 100 μm. Typically, the nervous system device can have a flexibility sufficient to allow rolling of the device into a roll having an outside rolled diameter less than about 1 mm, and often less than about 100 μm.

In one alternative, the flexible active layer can be a porous texture. In one embodiment, the porous texture can be at least one of a top surface of the flexible active layer, a bottom surface of the flexible active layer, and an outer surface of the communication module. In one aspect, the flexible active layer can be formed of a passivated thin porous silicon. In another aspect, the porous texture can include a plurality of holes from about 5 μm to about 15 μm in diameter. In one embodiment the porous texture can have a plurality of holes of about 10 μm in diameter. In some embodiments the holes in the porous texture can be substantially the same diameter in size or can be holes having different diameters.

The plurality of electrodes can be arranged on the flexible active layer. In one embodiment the electrodes can be coupled to a top surface of the flexible active layer. In another embodiment the electrodes can be coupled to a bottom surface of the flexible active layer. The number of electrodes in the plurality can depend upon desired performance criteria. However, generally, the nervous system interface device can include from about 2 electrodes to about 25 electrodes. In one embodiment the flexible active layer comprises from about 2 electrodes to about 10 electrodes. In another embodiment the flexible active layer can comprise about 4 electrodes, about 6 electrodes, about 8 electrodes, or about 12 electrodes. In some embodiments the electrodes can cover the entire surface of the flexible active layer. In other embodiments the electrodes can be scattered on the flexible active layer. The arrangement of the electrodes can be random or can be in an ordered array. In one embodiment the array can be along the coiled shape of the flexible active layer. In one example, the electrode array can be about 30 μm in width and 30 μm in length, although other dimensions can be functional. In other embodiments the array can be a 15 μm×15 μm electrode array or a 45 μm×45 μm electrode array. In specific embodiment, the electrode array can include 4 electrodes ranging in areas from 10 μm×10 μm to 100 μm×100 μm on a flexible substrate. The flexible substrate can have an effective average spring constant which forms a highly flexible substrate (k<k_{brain}), or a semi-flexible substrate (k˜k_{brain}), and in many cases k<<k_{brain} where k is at least 5% less than k_{brain}. In one embodiment, when the plurality of electrodes exhibit a higher level of flexibility, the electrode array can include a sucrose dissolvable polymer layer or coating to support the electrode array. The sucrose layer can then dissolve within tissue subsequent to insertion.

The electrodes can be composed of any biocompatible material or can be composed of a non-bio compatible material and coated in a biocompatible material. In one example, the electrodes can be composed of titanium which is a particularly biocompatible material which exhibits low toxicity and high corrosion resistance. In other examples, the electrodes can comprise platinum, platinum-iridium, titanium, gold, and combinations thereof. Conducting polymers can also be used. Since the electrodes sense the neuron firings in the 10 Hz-10 KHz range, they can be capacitively coupled to the extra-cellular neuron space. Thus, they can be insulated with insulating materials such non-conducting polymers, e.g. polyimide, parylene, and PDMS, as well as non-conducting ceramics such as glass and the like. The conductivity of the electrodes can also vary based on the electrode material.

The electrodes can be smooth or can have a textured surface. In one example, the electrodes can have a nano-textured surface. Suitable nano-textured surfaces can be obtained via any suitable technique such as, but not limited to, plasma etching, wet chemical etching, ion milling, and electrochemical forming. In one embodiment, the nano-textured electrodes can be used to maintain a relatively low resistance level (such as 1-4 kΩ in 1M saline solution) while maintaining a small electrode size. In another example, the electrodes can have sharp edges or rounded edges. The electrodes can be exposed portions of traces which are coated with a dielectric such as an insulating polymer (e.g. parylene).

The plurality of electrodes can be adapted to take neural measurements. The neural measurements can be taken from either the peripheral or central nervous system. The plurality of electrodes can be simultaneously or independently addressable. In one embodiment the electrodes can be independently addressable such that each electrode has an electrically independent trace which can be discriminated against other electrodes in the array. The plurality of electrodes can be exposed on outer surfaces of the device such that contact with surrounding tissue can provide desired electrical transmission and reception such as measuring neural signals. In one aspect, the plurality of electrodes can be coupled to a top surface of the flexible active layer.

The flexible active layer can further include distributed electronics. For example, amplifier transistors can be directly located under each electrode and send the transduced signal after amplification to the wireless communication circuit near the center of the flexible array. The power coil can be in any arrangement suitable for receiving a charge. In some embodiments the coil can be a circular or rounded shape coil. In other embodiments the coil can be a square shaped coil or polygonal shaped coil. The coil can be formed of a conductive material. Non-limiting examples of suitable coil materials can include titanium, stainless steel, tin, copper, gold, aluminum, tungsten, cobalt, nickel, polyimide, other biomaterials, and combinations thereof. In some embodiments, the power coil can be coated to improve corrosion resistance. In one embodiment the coating can be a titanium nitride coating or a parylene coating. The coil can be a wide variety of diameters. The diameter of coil can range from about 10 μm to about 150 μm. In one embodiment the diameter of the coil is about 50 μm, about 75 μm, about 100 μm, about 125 μm, or about 150 μm. In one embodiment, the power coil is a titanium coil on parylene with a 100 μm diameter.

The power coil can receive power from any wireless and/or inductive charging source. In one embodiment the power coil can receive power inductively from an exterior coil. FIG. 2b illustrates a plurality of nervous system interface devices 210-240 receiving wireless power from a radio frequency (RF) coil 250 of another device to power the nervous system interface devices 210-240. In another embodiment, the nervous system interface devices 210-240 can receive wireless power by rectifying 10-100 MHz signals, e.g. signals that have large biological penetration depth up to about 3 cm.

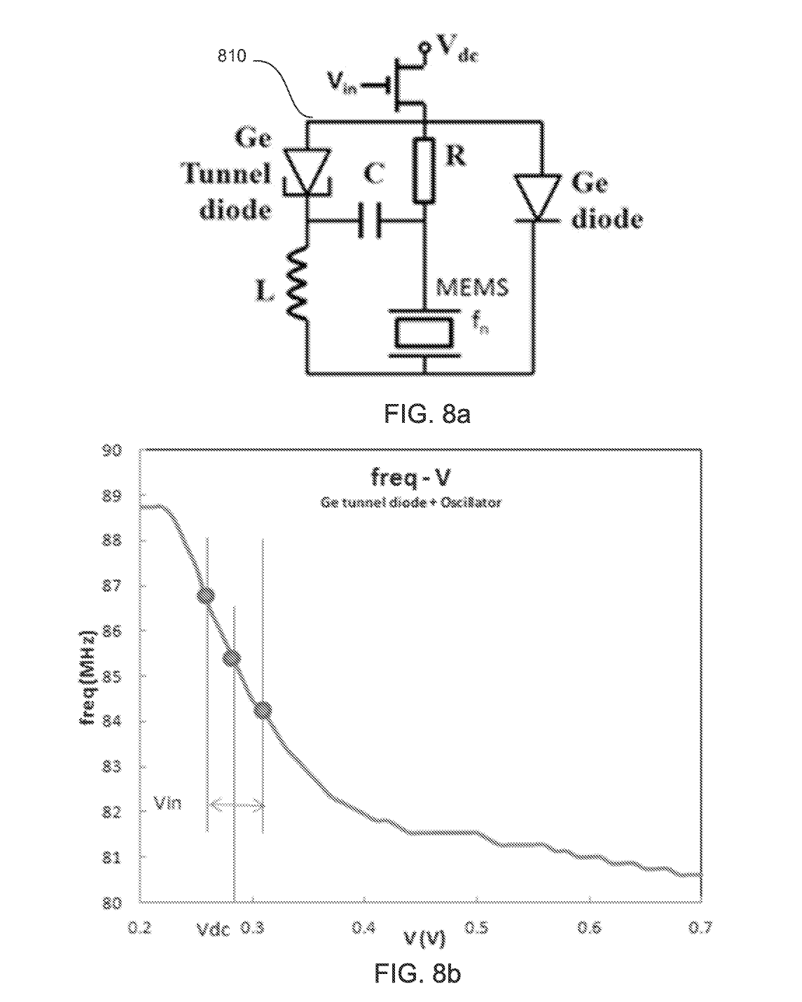

Signals from nearby neurons detected by the electrodes are generally around 10 μV and are amplified to 10-100 mV levels. At these amplified levels, they are transmitted to an external signal receiving system using a communication circuit such as the one shown in FIG. 8a. The communication module (FIG. 8a) can comprise a modulator that feeds the antenna and an amplifier bank that feeds the amplified neuron firing signals to the modulator. In one specific example, the communications module and the flexible active layer can each have a coiled shape which are coincident. Different electrode signals can be modulated at different frequencies to be simultaneously transmitted to the external system.

The antenna can be used to transfer radio frequency energy between an external transmitter and the electronic components of the nervous system interface device. The antenna can be a low profile antenna such as, a spiral micro-strip antenna, a planar inverted-F antenna (PIFA), a meandered PIFA, or a spiral PIFA. In one embodiment the antenna is a spiral PIFA that can be coupled to the power coil, communication module, and/or the flexible active layer. The antenna can be adapted to communicate wirelessly neural measurement information to another device that can be exterior of the patient. The antenna can also be adapted to receive electric signals from a separate communications device located exterior of the subject.

The antenna can be comprised of metallic traces on the back of the flexible substrate (electric dipoles) or can be a coil that forms a magnetic dipole. The antenna can send and receive radio frequencies within the range of 0-10 cm. The magnetic dipole antenna can also be used to power the array.

The wirelessly powered electrodes, circuitry, and/or modules can be used in combination with piezoelectric resonators and/or microelectromechanical systems (MEMS) resonators for signal amplification, data transmission, and/or addressing. In one example a high quality factor MEMS resonators (Q-100,000-1,000,000) can be used for low power micro watt (μW) frequency selection and addressing. In another configuration, the device can include tunnel diodes to reduce the size of the device and lower power transmission levels.

The nervous system interface device can be an entirely flexible device and can have two functional configurations. The configurations can include a rolled configuration and a substantially flat configuration, as well as, configurations in between the rolled and the flat configuration. Specifically, the device may only partially unroll within tissue depending on positioning, location and surrounding tissue resilience. When the nervous system interface device is in a rolled configuration the device can be rolled along either a horizontal or longitudinal axis. When the device is in a rolled configuration it can be capable of being inserted within a hollow shaft. The hollow shaft can be a tube or a hypothermic needle. The ability to roll the nervous system interface device permits it to be inserted into a subject with a minimal opening in the subject's protective coverings (skin and bone) and with minimal tissue damage. With respect to cranial implants the rolled configuration will permit the nervous system interface device to be implanted using a small hole in the skull. Moreover the rolled configuration permits the device to be inserted using a hypodermic needle into a specific area of the brain, rather than merely resting on the surface of the brain. Following insertion the nervous system interface device can either remain in a rolled configuration, partially unrolled, or substantially unrolled and lie in a substantially flat configuration. In one specific embodiment the nervous system interface device is rolled along a longitudinal axis for insertion into a hypodermic needle.

The nervous system interface device is designed to be a small device. The overall form factor of the device can vary, but can most often be substantially planar. Furthermore, the length and width can result in a planar aspect ratio of less than about 4:1, and in most cases less than about 2:1. As one example, the device has a linear dimension of about 10 μm to about 40 μm or a volume of 1,000-16,000 μm³. Similarly, the flexible active layer can be about 100 μm in diameter. The thickness of the device can also most often range from about 50 to 100 μm. The flexible active layer can also have planar dimensions from about 10 μm by 10 μm to about 100 μm by 100 μm.

In some embodiments the nervous system interface device can have holes/sieves that can help to reduce the occurrence of foreign body responses. In one embodiment when the array area is 100 μm×100 μm, the holes/sieves can be about 10 μm in size. Holes of 10 μm permit proteins and chemical to pass through the nervous system interface device unaffected and can reduce the body's reaction to the implant. Holes and sieves naturally result in textured electrode surfaces that increase their effective surface areas and further improve their ability to detect and sense neuron firings. In smaller array areas, holes and sieves may not be needed since the device itself is small enough to not evoke the foreign body response.

In one example, the nervous system interface device can provide a faster recovery time over traditional invasive electrode arrays largely because of a smaller implantation opening needed for the rolled device and needle. In another example, the nervous system interface device can have a reduced size compared to traditional invasive electrode arrays which can enable implanting of multiple nervous system interface devices and can permit studying neurons at different locations of the brain.

In some embodiments, the nervous system interface device can further comprise a flexible support member. The support member can be used to provide structure to the device and in some embodiments can enclose the flexible active layer, the power coil, the communication module, and the antenna. The size and shape of the flexible support member will depend on the particular application, intended target tissue, and desired device performance. The flexible support member can be composed of either non-biodegradable or biodegradable materials. Exemplary flexible substrates can include, but are not limited to, silicone, parylene, polydimethylsiloxane (PDMS), polyethylene terephthalate, polyethylene, thin polycarbonates, polyimides, and the like. In one embodiment the dissolvable polymeric matrix comprises at least one of parylene and a polydimethylsiloxane (PDMS) substrate. In some embodiments, the flexible substrate can be a dissolvable polymeric matrix. When the flexible support member comprises a dissolvable polymeric matrix, the polymer matrix can increase structural integrity of the flexible support member for a selected period of time before it dissolves after insertion into the nervous system. Although a wide variety of materials for the dissolvable polymeric matrix can be suitable, non-limiting examples include at least one of sugar, starch, PLA, PGA, albumin, certain waxes, copolymers thereof, and the like. The dissolvable polymeric matrix can be useful to provide temporary structural rigidity to the device during manipulation and/or early stages of implantation.

Furthermore, the dissolvable polymeric matrix can optionally include biologically active materials which are released into surrounding tissue and provide specific biological, analgesic, or other pharmacokinetic benefits. Non-limiting examples of such active materials can include at least one of growth hormones, dexamethasone, minocycline, antibiotics, local anesthetics, and the like. In some embodiments, the nervous system interface device can further comprise a flexible polymeric battery or several flexible polymeric batteries. The flexible polymeric battery can be a thin battery and can be a single use or re-chargeable battery. The flexible polymeric battery can be used to store power and charge the nervous system interface device electronically and telemetry. In one embodiment the flexible polymeric battery will be located under the antenna. In other embodiments the flexible polymeric battery can be coupled to the power coil, configured to receive energy from the power coil, and be configured to store received energy in a cell of the flexible polymeric battery.

In some embodiments the energy is stored in two or more cells of the flexible polymeric battery. In other embodiments, the nervous system interface device can further comprise distributed electronics. In some embodiments these electronics can interconnect the plurality of electrodes. In one embodiment the distributed electronics can be coupled to the flexible active layer.

In another embodiment, the nervous system interface device can be used as an in-situ sensor to monitor electrode impedance spectra as a function of time. In one example, the impedance spectra can be analyzed using a Cole-Cole plot. In another embodiment, the nervous system interface device can monitor electrode-to-electrode and/or electrode-to-body impedances to determine when the electrode surfaces are blocked or can conduct electrical signals. In another embodiment, the nervous system interface device can monitor electrode-to-electrode and/or electrode-to-body impedances to determine an ionic nature and/or electronic nature of foreign body response (FBR) cells and molecules that deposit on the electrodes of the nervous system interface device.

A foreign body response (FBR) using neural sensor probes for monitoring and measuring electrical signals or magnetic signals of animal brains and/or human brains can increase or decrease based on type and configuration of a neural sensor probe. In one example, the FBR can change based on the size of the neural sensor probe, a size of an electrode array of the neural sensor probe, a geometry of the neural sensor probe, a material of the neural sensor probe, a surface morphology of the neural sensor probe, mechanical impedance or flexibility of the electrode array, surface material characteristics of the electrode array (such as different metals, semiconductors, conducting polymers with glassy surface, conducting polymers with textured surfaces, and so forth), and/or whether the neural sensor probe uses a wired connection or wireless connection to communicate information.

The FBR can increase significantly when the neural sensor probe uses a wired connection with the wires going through various biological boundaries of an animal or human. The FBR can decrease significantly when the neural sensor probe is relatively small, such as 10-40 μm in linear dimensions or a volume of 1,000-16,000 μm³ and can be reduced when electrodes with larger surfaces include holes and/or sieves along the surface of the electrodes and optionally across the entire device. Although specific dimensions can vary the holes can range from 0.5 μm to about 40 μm, such as 10 μm sized holes. The holes and/or sieves can enable FBR proteins and chemicals to pass through the holes of the electrode surfaces without affecting the FBR proteins and chemicals.

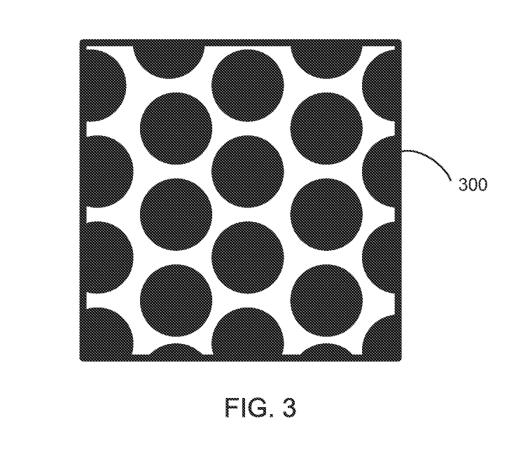

For example, FIG. 3 illustrates that a top surface and/or a bottom surface of the flexible coil (as in FIG. 2) can have a porous texture or have sieves 300 to reduce a FBR. In one embodiment, a flexible substrate with sieves and porosity can be used to reduce the FBR and maximize extra-cellular neuron transduction. In another embodiment, the flexible coil can include a dissolving polymeric substrate which adds a selected structural integrity to the flexible coil for a selected period of time, such as until a selected time after the nervous system interface device has been inserted into a patient. In one example, different polymeric substrates, electrode materials, surface texturing, and/or surface porosity can be used to enhance extra-cellular signal detection, reduce FBR, and/or extend an active lifetime of the nervous system interface device.

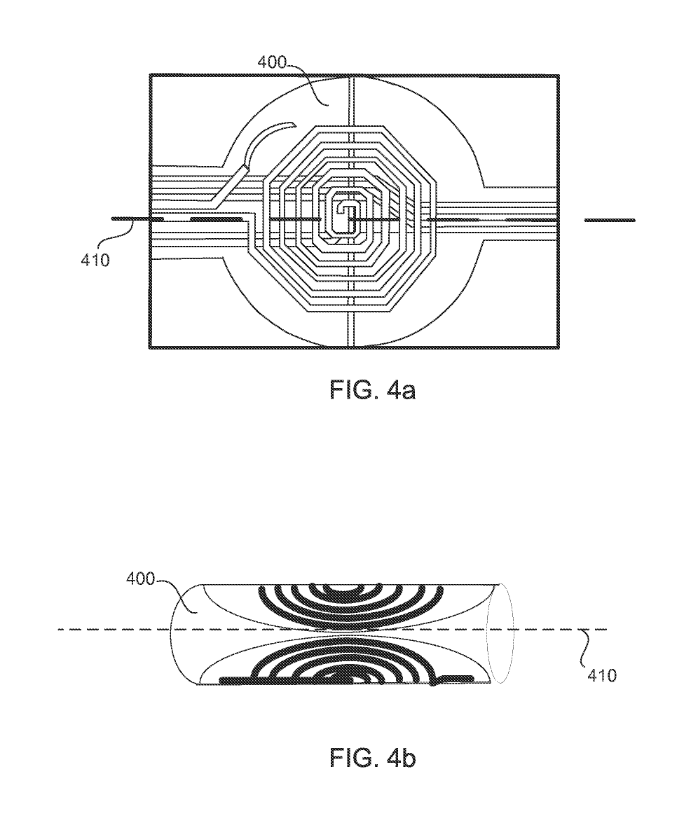

FIGS. 4a and 4b illustrate that the nervous system interface device 400 can have an unrolled configuration (FIG. 4a) and a rolled configuration (FIG. 4b). FIG. 4a illustrates an unrolled configuration where the nervous system interface device 400 is substantially flat or curved to engage a nervous system membrane surface of a patient. FIG. 4a further illustrates that the nervous system interface device 400 can have a longitudinal axis 410 to roll the nervous system interface device 400 into a rolled configuration as in FIG. 4b. FIG. 4b illustrates a rolled configuration wherein the nervous system interface device 400 rolled along a longitudinal axis 410 for insertion into a hypodermic needle. FIG. 4a also illustrates interconnects between different parts of the flexible electrode arrays. These interconnects power distributed devices and carry the amplified signals from one part of the array to another part for further processing and transmission or control.

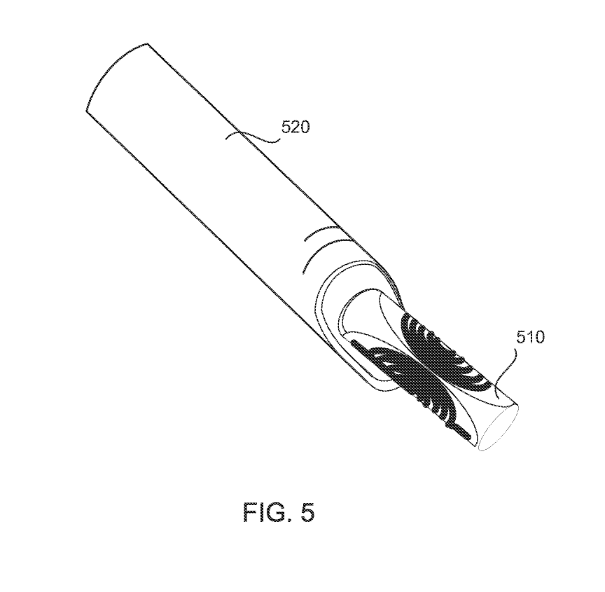

FIG. 5 illustrates a nervous system interface device 510 in a rolled configuration (as in FIG. 4b) inserted into a hypodermic needle 520. In one embodiment, the nervous system interface device 510 can be used model a motor brain machine interface. In one example, a plurality of nervous system interface devices 510 can be implanted in a primate brain and in multiple areas of the primate brain. Traditional implantable electrodes are relatively large (in excess of 1 cm²) and implanting the electrode into a cranium of an animal or human can require a major brain surgery, such as a craniotomy. By contrast the nervous system interface device 510 presented herein can be implanted less invasively than traditional procedures by burring one or more holes into the cranium of an animal or human and injecting the nervous system interface device 510 using the hypodermic needle 520. One advantage of less invasive implantation procedures is that the nervous system interface device 510 can be implanted as an outpatient procedure and with electrodes that last a considerably longer period of time than the existing arrays. It is expected that such devices can provide up to 50 years of useful neural interface functionality. The long-term placement can allow for prolonged monitoring and/or treatment in subjects that have chronic neurological disorders.

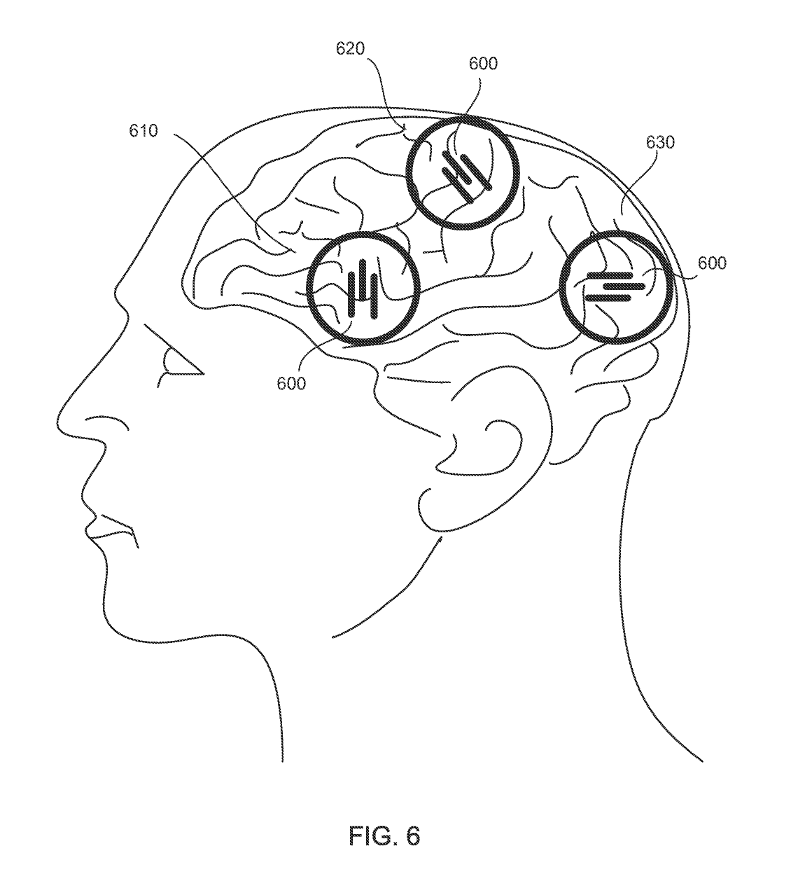

FIG. 6 illustrates that a nervous system interface device 600 can record signals (such as sensory and motor signals) in multiple brain areas 610, 620, and 630. This allows practitioners to record a wide area of the brain or multiple areas of the brain. A plurality of nervous system interface devices 600 can be distributed in one or more brain areas (item 600 is not shown to scale for clarity). In one embodiment, the recorded signals can be correlated with overt arm movement in motor areas and/or arm palpation in sensory areas of an animal or human. Brain area 610 is generally associated with the Broca's area to allow recording or controlling speech and initiates speech for speech based neuroprosthetics. Brain area 620 is associated with the primary motor cortex which controls skeletal muscle function for movement based neuroprosthetics. Brain area 630 is the visual cortex receive visual input so as to provide for vision based neuroprosthetics. Premotor areas, supplementary motor areas, parietal reaching areas and other brain areas can also be accessed by the nervous system interface devices presented herein.

In one embodiment, the nervous system interface device can be implanted into the brain or tissues of living animals. In one example, the nervous system interface device can be implanted into the brain or tissues of 7^{th} or 8^{th} grade biology/science class animals and used in observing real-time brain activities or electromyogram signal on a display. In another example, the nervous system interface device can be implanted into the brain or tissues of a plurality of frogs in a science museum to enable an individual to observe various frog activities and underlying brain neuron firing patterns for the various frog activities on a display. In another example, the nervous system interface device can enable access to neuronal signals and the monitoring of bio-electrical signals of animals by high-school students, engineering faculty, and other parties. In another example, the nervous system interface device can monitor a strength of the neural firings. Selectable transponders can be used to record neural firing wirelessly. One advantage of the nervous system interface device is to provide reliable interfaces to monitor signals of central nervous systems of animals and humans. The device can also be used to monitor signals of the peripheral nervous system. In one embodiment, the nervous system interface device can be placed adjacent to a peripheral nerve.

In another embodiment, the nervous system interface device or wireless electrodes of the nervous system interface device can include other sensors such as pressure sensors, temperature sensors, bio-chemical sensors, and/or gas sensors. In one example, the other sensors can be used to correlate neuron firings with in-situ neurotransmitter release, minute temperature fluctuations, blood/fluid flow, and/or pressure changes. These nervous system interface devices can thus be additionally used for treatment and study of nervous system ailments including chronic central nervous system ailments such as, but not limited to, Parkinson's, Alzheimer, epilepsy, early seizure detection, and the like.

For brain-machine interface (BMI) applications, a much higher bandwidth neural control signal may be required. This involves many more channels from multiple brain areas related to movement. This simply cannot be done through a non-wireless approach due to connectorization. Even systems that route wires to implanted titanium canisters may be too limiting. The nervous system interface device provokes a minimal immune response, and can be widely deployed throughout the brain. Thus, the nervous system interface device can enable full control of all 28 degrees of freedom in the human arm.

In vision and auditory prosthetics, the nervous system interface devices presented herein can have major impact by enabling access to neuronal activities in the auditory and visual cortext to directly monitor the effected of cochlear and retina implants. Such systems are currently geometry limited with respect to how many independent channels they can support, which this device could alleviate.

In one embodiment, the nervous system interface device can be approximately the size of a grain of rice and can be inserted into different parts of a brain of an animal to provide access to neuronal activities simultaneously across the central nervous system. One advantage of the nervous system interface device shaped and sized to be injected using a hypodermic needle is to enable the implantation of the nervous system interface device into location that traditionally were not reachable with traditional electrodes, such as the primary motor cortex (including premotor areas, supplementary motor areas and parietal reaching areas). Another advantage of the nervous system interface device is to reduce an invasiveness of implanting electrode into the cranium of an animal or human. For example, the nervous system interface device can be inserted into the cranium of an animal or human using a hypodermic needle and the implantation procedure can be an outpatient procedure since only small holes in the skull would be used to implant the nervous system interface device.

Traditional implantable electrode devices can be limited to 4 channels due to connector sizes or a density of available hermetic feed through connectors of the implantable electrode devices. One advantage of the nervous system interface device presented herein is that a channel count of an electrode of the nervous system interface device is not limited by a connector size or a density of available hermetic feed through connectors.

In the area of neuroprosthetic systems, the injectable nervous system interface device can increase a bandwidth of neural controls signals by over an order of magnitude and provide access to a wide variety of brain areas. In another example, the injectable nervous system interface device can measure neural signals used to predict an intended motion of an arm of an animal or human by correlating neural signals with recorded movements and palpations of the animal or human.

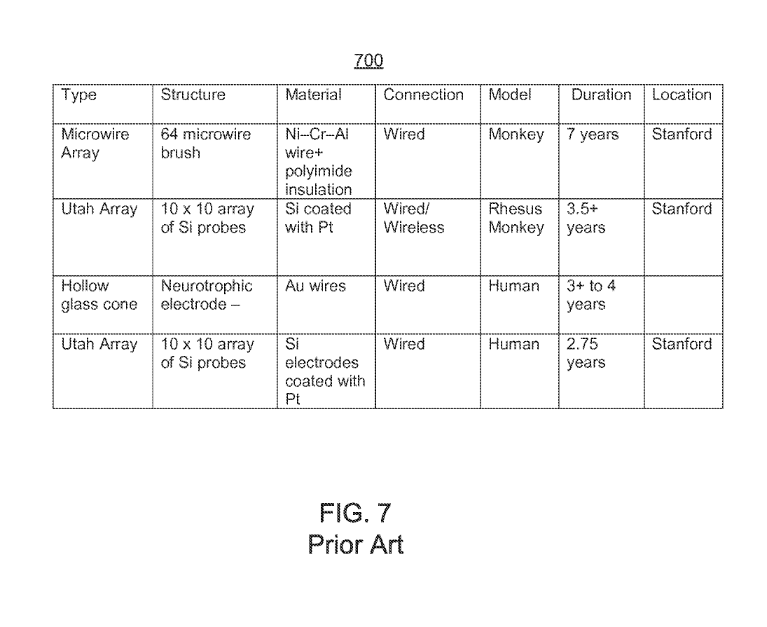

FIG. 7 is a table 700 of prior art electrode devices with different configurations of electrode arrays. The table includes a type of electrode array, a configuration or structure of the electrode array, a material used for the electrode array, a type of connection used to interface with the electrode devices, a type of animal used with the electrode devices, a duration the electrode devices was used, and a location the electrode devices was used.

FIG. 8a illustrates a low power frequency-selectable and addressable FM transmitter 810 for neural recording. In one embodiment, the transmitter 810 can be an analog transmitter that operates at a low power amount, such as 60 μW. In another embodiment the transmitter 810 can be a compact transmitter with a size of 50 μm×50 μm area. In one embodiment, the transmitter 810 can be powered by a polymeric rechargeable battery. In one embodiment, the transmitter 810 can transmit extracellular signal levels are around a few mV. In another embodiment, the transmitter 810 can include front end electronics with a voltage gain to provide mV range signals for transmission. In another embodiment, the transmitter 810 can include a very low power and small area (such as 100 μm×100 μm) germanium (Ge) tunnel diode and/or a microelectromechanical (MEMs) resonator for stable operation and frequency selection. FIG. 8b illustrates a graph of a frequency versus voltage level of the transmitter, as in FIG. 8a.

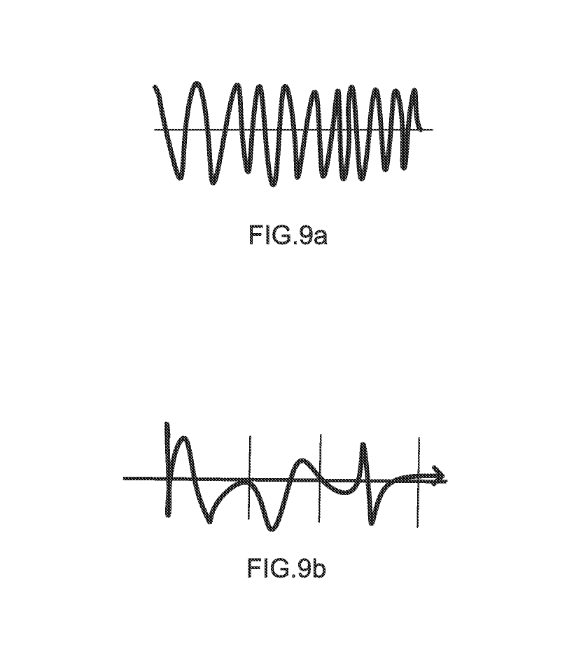

In one embodiment, when a nervous system interface device detects single unit bipolar waveforms, a signal to noise ratio (SNR) can be quantified over time. In one example, single units can be sorted by using a first few principal components of the waveforms that cross a selected root mean square (RMS) threshold value and clustered using a Gaussian model. The SNR is defined by a peak to peak height of a largest isolated waveform on a given channel over an average RMS activity on a channel. FIGS. 9a and 9b depict graphs of different neural signals, send and receive, respectively.

In one embodiment, when single units cannot be obtained during a period the nervous system interface device records neural signals, a multiunit activity can be used to gather information. In one embodiment, the nervous system interface device can measure crossing events as well as signal power in a spiking band (˜50-500 Hz) with simple measures of behavior, e.g. monitor periods of activity and inactivity. In one example, power in the spiking band inversely correlates with power in a beta band (10-30 Hz) and can indicate neural activity over a large area of a brain.

In one embodiment, the nervous system interface device can be implanted into an animal or human with a recording cylinder used for single unit electrophysiology using neurosurgical procedures. In one example, the neurosurgical procedures can include using a protective enclosure attached to the skull with methyl methacrylate. Within the enclosure, the brain can be accessed by drilling a small burr hole. A hypodermic needle with one or more nervous system interface devices can be inserted to implant several neural recording devices into the central nervous system of the animal or human. Diameter of the hole can vary depending on the needle outer diameter, but typically range from about 0.1 mm to about 5 mm. In another example, two burr holes can be made over a primary motor cortex and a primary sensory cortex to record neural signals over one or more central nervous system areas separated by a distance too large to be covered with one microelectrode array. In another example, after one or more nervous system interface devices have been implanted, the burr holes can be resealed using methyl methacrylate or other biologically suitable agent.

When the nervous system interface devices have been implanted, an RF coil can be placed above the implanted area to power the nervous system interface devices neural signals while neural measurements are taken and/or recorded at selected times, such as daily. In one embodiment, neural signals or an SNR of the signals transmitted by the nervous system interface device can be monitored and/or tracked over a selected time interval, such as on a hourly basis, a daily basis, a weekly basis, or longer. Thus the nervous system interface device can also include onboard memory which records and stores collected information.

In one example, the neural signals can provide useful information, such as neural information related to arm movements of an animal or human. In one embodiment, another device can track the arm or hand motion using a passive, infrared motion capture system. In another embodiment, the arm or hand motion can be correlated with neural measurements take using the nervous system interface device and the correlation can be used to tune or calibrate neural signals taken using nervous system interface device. In one example, the depth of the tuning can vary from moving the arm or hand a few degrees to moving the arm or hand around 360 degrees. In another example, the degrees of movement can be compared with variance in the neural signals. In this example, the nervous system interface device or another computing device can correlate individual neurons of the animal or human with x, y, and/or z positions of the hand to determine the amount of variance in a firing rate of a neuron.

It should be understood that many of the functional units described in this specification have been labeled as modules, in order to more particularly emphasize their implementation independence. For example, a module can be implemented as a hardware circuit comprising custom VLSI circuits or gate arrays, off-the-shelf semiconductors such as logic chips, transistors, or other discrete components. A module can also be implemented in programmable hardware devices such as field programmable gate arrays, programmable array logic, programmable logic devices or the like.

Modules can also be implemented in software for execution by various types of processors. An identified module of executable code can, for instance, comprise one or more physical or logical blocks of computer instructions, which can, for instance, be organized as an object, procedure, or function. Nevertheless, the executables of an identified module need not be physically located together, but can comprise disparate instructions stored in different locations which, when joined logically together, comprise the module and achieve the stated purpose for the module.

Indeed, a module of executable code can be a single instruction, or many instructions, and can even be distributed over several different code segments, among different programs, and across several memory devices. Similarly, operational data can be identified and illustrated herein within modules, and can be embodied in any suitable form and organized within any suitable type of data structure. The operational data can be collected as a single data set, or can be distributed over different locations including over different storage devices, and can exist, at least partially, merely as electronic signals on a system or network. The modules can be passive or active, including agents operable to perform desired functions.

Further presented herein is an intracranial electrode system comprising a shaft, an opening in the shaft, and an intracranial electrode. The shaft can be a tube or hypodermic needle as described above. The shaft can be sized and shaped to be inserted into a cranial opening of a patient. The opening in the end of the shaft can be sized and shaped to be positioned subcutaneously adjacent to a patient's cranial membrane and receive an intracranial electrode. In one example, the shaft opening can be angled such that edges of the shaft form a sharp penetrating leading edge (as generally illustrated in FIG. 5). The intracranial electrode can be sized and shaped to be rolled along a longitudinal axis and adapted to be inserted into the opening of the shaft. The system can further comprise a wireless energy transfer device adapted to wirelessly transfer energy to the intracranial electrode. The system can also comprise a wireless communication device adapted to wirelessly receive neural measurement information from the intracranial electrode. The intracranial electrode can be as described above as a nervous system interface device. The wireless energy transfer device can be any device capable of transferring energy wireless, e.g. inductively. The wireless communication device can be any external device designed to receive neural measurements.

Additionally presented is a method for taking a neural measurement. The method can comprise inserting a flexible electrode into a delivery shaft, wherein the flexible electrode is sized and shaped to be rolled along a longitudinal axis for insertion into the delivery shaft. The delivery shaft can be inserted into an opening of a patient adjacent target tissue. The flexible electrode can then be discharged from the shaft into the opening of the patient. Although the electrode can be pre-charged, wireless power can be transferred from a power transfer device to the flexible electrode. Neural measurements can be acquired using the flexible electrode. At least a portion of these neural measurements can be wirelessly communicated from the flexible electrode to a receiving device. In one embodiment, the flexible electrode used in the method can be an intracranial electrode and the opening is an intracranial opening. In a second embodiment, the opening is adjacent a peripheral nerve. The electrode can be inserted into or adjacent to the peripheral nerve. The method above incorporates the nervous system interface device comprised of a plurality of flexible electrodes and the delivery shaft (tube or a hypodermic needle) as discussed above. The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A nervous system interface device, comprising:
a flexible active layer including a plurality of electrodes that are positioned to take neural measurements of a nervous system;
a power coil electrically coupled to the flexible active layer and configured to receive wireless power from a power transfer device;
a communication module electrically coupled to the flexible active layer and configured to receive power from the power coil; and
an antenna adapted to wirelessly communicate neural measurement information to another device;
wherein the nervous system interface device has two functional configurations:
a rolled configuration, wherein the electrode device is rolled along a longitudinal axis for insertion into a hypodermic needle; and
an unrolled configuration, wherein the electrode device is substantially flat.

2. The device of claim 1, further comprising:
a flexible support member encompassing the device and includes a dissolvable polymeric matrix for providing a selected structural integrity to the flexible support member for a selected period of time and which dissolves after insertion into the nervous system.

3. The device of claim 2, wherein the dissolvable polymeric matrix comprises at least one of a parilyne substrate and a polydimethylsiloxane (PDMS) substrate.

4. The device of claim 2, wherein the dissolvable polymeric matrix further includes at least one of growth hormones, dexamethasone, and minocycline.

5. The device of claim 1, further comprising a flexible polymeric battery that is:
coupled to the power coil;
configured to receive energy from the power coil; and
configured to store the energy in one or more cells of the flexible polymeric battery.

6. The device of claim 5, wherein the energy is stored in two or more cells.

7. The device of claim 1, wherein at least one of a top surface of the flexible active layer, a bottom surface of the flexible active layer, and an outer surface of the communication module have a porous texture.

8. The device of claim 6, wherein the porous texture includes a plurality of holes from about 5 μm to about 15 μm in diameter.

9. The device of claim 1, wherein the flexible active layer has a coiled shape.

10. The device of claim 1, wherein the plurality of electrodes are arranged in an array along the coiled shape.

11. The device of claim 9, wherein the array of plurality of electrodes is about 30 μm in width and 30 μm in length.

12. The device of claim 1, wherein the plurality of electrodes are independently addressable.

13. The device of claim 1, wherein the plurality of electrodes are coupled to a top surface of the flexible active layer.

14. The device of claim 1, wherein the flexible active layer further includes distributed electronics.

15. The device of claim 14, wherein the distributed electronics interconnect the plurality of electrodes.

16. The device of claim 1, wherein the communications module and the flexible active layer each have a coiled shape which are coincident.

17. The device of claim 1, wherein the plurality of electrodes are adapted to take neural measurements.

18. The device of claim 1, wherein the device has a linear dimension of about 10 μm to about 40 μm or a volume of 1,000-16,000 μm³.

19. The device of claim 1, wherein the flexible active layer has an area from about 10 μm by 10 μm to about 100 μm by 100 μm.

20. An intracranial electrode system, comprising:
a shaft sized and shaped to be inserted into a cranial opening of a patient;
an opening at an end of the shaft, the opening being sized and shaped to:
be positioned subcutaneously adjacent to a patient's cranial membrane; and
receive an intracranial electrode; and
an intracranial electrode sized and shaped to be rolled along a longitudinal axis and adapted to be inserted into the opening of the shaft.

21. The system of claim 20, further comprising a wireless energy transfer device adapted to wirelessly transfer energy to the intracranial electrode.

22. The system of claim 20, further comprising a wireless communication device adapted to wirelessly receive neural measurement information from the intracranial electrode.

23. A method for taking a neural measurement, comprising:
inserting a flexible electrode into a delivery shaft, wherein the flexible electrode is sized and shaped to be rolled along a longitudinal axis for insertion into the delivery shaft;
inserting the delivery shaft into an opening of a patient;
discharging the flexible electrode from the shaft into the opening of the patient;
transferring wireless power from a power transfer device to the flexible electrode;
taking a neural measurement using the flexible electrode; and
wirelessly communicating the neural measurement from the flexible electrode to a receiving device.
